(19) United States
(12) Patent Application Publication
(10) Pub. No.: US 2026/0369362 A1
(43) Pub. Date: Nov. 19, 2026

(54) SYSTEMS AND METHODS FOR PHENOTYPIC BASED DRUG DESIGN

(71) Applicant: PGI Drug Discovery LLC, Jacksonville, FL (US)

(72) Inventor: Daniela BRUNNER, Bronx, NY (US)

(73) Assignee: PGI Drug Discovery LLC, Jacksonville, FL (US)

(21) Appl. No.: 19/135,325

(22) PCT Filed: Dec. 5, 2023
(86) PCT No.: PCT/US2023/082421

§ 371 Date: Jun. 3, 2025

Related U.S. Application Data
(60) Provisional application No. 63/386,170, filed on Dec. 5, 2022.
(60) Provisional application No. 63/430,471, filed on Dec. 6, 2022.

(51) Int. Cl. G16C 20/70; G16C 20/30; G16C 20/50; G16H 20/10

(52) U.S. Cl. CPC G16C 20/70; G16C 20/30; G16C 20/50; G16H 20/10

(57) ABSTRACT

A method for configuring a structural-phenotypic model can involve obtaining observational data acquired from non-human animals during trials in which the non-human animals were administered at least one of a set of compounds. The method can include obtaining structural data for the set of compounds. The method can include generating a structural dataset using the structural data by mapping the structural data to a first space. The method can include generating a phenotypic dataset using the observational data by mapping the observational data to a second space. The method can include configuring a machine learning model, using the phenotypic dataset and the structural dataset, to map between encoded phenotypic data and encoded structure data.

### CROSS-REFERENCE TO RELATED APPLICATIONS

This disclosure claims priority to U.S. Provisional Application No. 63/386,170, titled “Phenotypic-Based Drug Design,” filed Dec. 5, 2022, and U.S. Provisional Application No. 63/430,471, titled “Phenotypic-Based Drug Design,” filed Dec. 6, 2022, the contents of which are incorporated herein in their entirety.

### TECHNICAL FIELD

The present disclosure relates to devices, systems, and methods for capturing, analyzing, and augmenting behavioral and physiological data of subjects. In particular, the subjects can be non-human animals and the behavioral and physiological data can be captured during a trial in which a compound is administered.

### BACKGROUND

The evaluation of compounds (e.g., as part of a drug development program) can involve non-human animal trials. Such animal trials can provide the safety and efficacy data necessary to support subsequent human trials.

Behavioral assessments can be an important component of such trials. The effects of experimental substances on animal behavior can provide information about the potential clinical effects of the experimental substances on human subjects. For example, the discovery that chlorpromazine produces differential effects on avoidance and escape behavior in animals encouraged the evaluation of the behavioral effects of other experimental antipsychotic drugs.

A behavioral assessment can include comparing the behavior of an animal treated with a compound to the normal behavior of the animal (or another animal). A behavioral phenotype can be generated from a set of monitored behaviors. The behavioral phenotype can correlate the administration of the compound with the behavior or physiology of the animal during the trial. Behavioral platforms can automatically collect observational data for the generation of such behavioral phenotypes. As compared to manual collection of observational data, automatic data collection can be more complete, repeatable, reliable, and accurate.

Further, drug discovery involving polypharmacology, such as for psychiatry research and mental health indications, may benefit from in vivo phenotypic drug discovery (iPDD) approaches. An iPDD platform can be used for polypharmacology against known multi targets or in a target-agnostic manner as the organism used for drug screening acts as an amplifier of all compound's actions, providing a comprehensive drug profile. iPDD platforms can include high-throughput screens based on drosophila, zebrafish, and mice. Although iPDD platforms may provide information (potentially including physiological information, such as heart rate or the like, and electrophysiological information, such as electroencephalography (EEG) measurements) regarding blood-brain barrier (BBB) crossing, potency, efficacy, therapeutic window, and side effect profile all at once, the comprehensive data they produce has yet to be modeled and integrated with medicinal chemistry in a way that enables de novo drug design. For example, while structural data may be readily available, phenotypic data, and corresponding standardized behavioral readouts, are not so available. iPDD platforms can provide insights, including for phenotypic data, that may not be properly leveraged with conventional techniques. Phenotypic approaches for AI-based drug design promise to bring new insights and accelerate drug discovery for CNS disorders.

Embodiments of the present disclosure provide a comprehensive model that links complex chemical structural information with phenotypic data to accelerate drug discovery using machine learning.

Additionally, iPDD data and methods allow identification of drug clusters in an unsupervised manner, such that that the algorithm does not know about the a priori therapeutic classes each group belongs to, or even the compounds' structures. The grouping(s) of drugs identified by this method, especially the profiles of different doses of some drugs (e.g., ketamine), would not be possible with data solely based on either chemical structure or receptor binding.

Phenotypic screening can enable characterization of the complete gamut of behavioral effects of reference drugs and a data-driven, target-agnostic comparison with novel compounds. The potential of in silico analysis based on behavioral phenotyping can be leveraged in many drug discovery applications, such as the mining of data from iPDD screened compounds libraries, or analysis of novel drug candidate analogs, to speed up the lengthy drug discovery process. Furthermore, using animal models of disease opens opportunities to explore phenotypic screening for rare disorders.

The potential of in silico analysis based on behavioral phenotyping can be leveraged in many drug discovery applications, such as the mining of data from iPDD-screened compounds libraries, or analysis of novel drug candidate analogs, to speed up the lengthy drug discovery process. Furthermore, using animal models of disease opens opportunities to explore phenotypic screening for rare disorders.

However, basing de novo drug design solely on the structure plus one simple number representing desired functionality (such as binding to a receptor) carries the risk of over-simplifying structure-receptor relationships.

### SUMMARY

Certain embodiments of the present disclosure relate to systems and methods for configuring a structural-phenotypic model.

The disclosed embodiments involve obtaining observational data acquired from non-human animals during trials in which the non-human animals were administered at least one of a set of compounds. The disclosed embodiments can involve obtaining structural data for the set of compounds. The disclosed embodiments can involve generating a structural dataset using the structural data by mapping the structural data to a first space. The disclosed embodiments can involve generating a phenotypic dataset using the observational data by mapping the observational data to a second space. The disclosed embodiments can involve configuring a machine learning model, using the phenotypic dataset and the structural dataset, to map between encoded phenotypic data and encoded structure data.

In some embodiments, the set of compounds can include one or more of a predicted therapeutic class of an antidepressant, analgesic, anxiolytic, antipsychotic, cognitive enhancer, hallucinogen, anticonvulsant, mood stabilizer, or psychostimulant and ADHD medication, anesthetic, or hypnotic when administered to humans. In some embodiments, the set of compounds can include one or more reference drugs tested in clinical trials. In some embodiments, the set of compounds can include one or more preclinical compounds.

In some embodiments, mapping the structural data can include determining a pairwise distance between first structure data and second structure data in the structural data. In some embodiments, the first structure data may correspond to a first compound in the set of compounds and the second structure data may correspond to a second compound in the set of compounds. Some disclosed embodiments may include mapping the first compound to a location in the first space using the pairwise distance. In some embodiments, the pairwise distance may include a Tanimoto score.

In some embodiments, mapping the observational data to the second space further includes determining a pairwise distance using first observational data and second observation data in the observational data. In some embodiments. the first observation data can correspond to a first compound and the second observational data can correspond to a second compound. Some embodiments may include associating the first compound with a location in the second space using the pairwise distance.

In some embodiments, the pairwise distance may include at least one of a binary classifier output or a similarity distance. In some embodiments, the observational data may be mapped to the second space using a phenotypic machine learning model.

Some disclosed embodiments involve obtaining, for a subset of the set of compounds, therapeutic class labels, and training the phenotypic machine learning model using the subset and the therapeutic class labels. Some disclosed embodiments involve generating a predicted phenotypic datum using the machine learning model and a structural datum for a trial compound. Some disclosed embodiments include providing a predicted therapeutic class based on the predicted phenotypic datum.

In some embodiments, the phenotypic dataset involves extracting from the observational data for an animal instant behavioral features describing at least one of size, shape, posture, or movement of the animal. In some embodiments, generating the phenotypic dataset involves extracting from the observational data for an animal higher order behavioral features describing at least one of motifs, sequences, or domains of behavioral function of the animal. In some embodiments, generating the phenotypic dataset involves obtaining a first phenotypic signature for a first compound using first observation data corresponding to the first compound. In some embodiments, the first phenotypic signature includes therapeutic effect probabilities of the compound when administered to a human.

In some embodiments, obtaining the first phenotypic signatures involves normalizing the first phenotypic signature using a phenotypic signature of a control compound. In some embodiments, generating the phenotypic dataset involves obtaining phenotypic signatures for a first compound that correspond to minimal and maximal therapeutic doses of the first compound. In some embodiments, generating the phenotypic dataset involves obtaining phenotypic signatures for a first compound that correspond to minimal and maximal tolerated doses.

In some embodiments, structural data may include a two-dimensional chemical structure model. In some embodiments, the structural data may include a three-dimensional chemical structure model. In some embodiments, the structural data may include a chemical sub-structure model. In some embodiments, the structural data may include electrostatic data. In some embodiments, the structural data may include stereochemistry data.

### BRIEF DESCRIPTION OF DRAWINGS

The accompanying drawings, which are incorporated in and constitute a part of this specification, illustrate several embodiments and, together with the description, serve to explain the disclosed principles. In the drawings:

FIG. 1 depicts a system for acquiring (and optionally analyzing) observational data concerning a non-human animal, consistent with disclosed embodiments.

FIG. 2 illustrates a table of exemplary behavioral phenotypic signatures, consistent with disclosed embodiments.

FIG. 3 depicts a block diagram of a process for predicting a behavioral phenotype of a compound using the structure of the compound, consistent with disclosed embodiments.

FIG. 4 illustrates exemplary chemical structures, consistent with disclosed embodiments.

FIG. 5 depicts an exemplary process for mapping structural data to a structure representation space, consistent with disclosed embodiments.

FIG. 6 depicts locations of exemplary compounds in a structure representation space, consistent with disclosed embodiments.

FIG. 7 depicts an exemplary process for mapping phenotypic data to a behavioral phenotype representation space.

FIG. 8 depicts locations of exemplary compounds in a behavioral phenotype representation space, consistent with disclosed embodiments.

FIG. 9 depicts a process for generating a structural-phenotypic mapping using distances, consistent with disclosed embodiments.

FIG. 10 depicts an exemplary mapping between a structural representation space and a behavioral phenotype representation space, consistent with disclosed embodiments.

FIG. 11 depicts a process for generating novel compounds based on structural and phenotypic data, consistent with disclosed embodiments.

FIG. 12 depicts a process for generating novel compounds based on structural and phenotypic data, consistent with disclosed embodiments.

FIG. 13 depicts a computing system for performing the disclosed embodiments.

### DETAILED DESCRIPTION

Reference will now be made in detail to exemplary embodiments. discussed with regard to the accompanying drawings. In some instances, the same reference numbers will be used throughout the drawings and the following description to refer to the same or like parts. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that some embodiments may be utilized and that changes may be made without departing from the scope of the disclosed embodiments. Thus, the materials, methods, and examples are illustrative only and are not intended to be necessarily limiting.

Machine learning models consistent with disclosed embodiments can predict the effect(s) of a compound in humans based on the effect(s) of the compound in non-human animals. Such predictions of the effect(s) of a compound in humans can support drug development by providing an early indication of potentially therapeutic compounds. For the example of complex CNS disorders, phenotypic screens and associated AI methods (such as iPDD platforms) may be useful for leveraging phenotypic and/or structural data, which may include large amounts of data from one or more trials. Linking structural data for compounds with phenotypic data for the compounds, and predicting and/or classifying behavior for the compounds, may be an unconventional method for drug development. Such methods may be useful for generating novel compounds and understanding how modifications to existing compounds can affect predicted behaviors associated with the compounds (e.g., behavioral uses which can be therapeutic). The disclosed embodiments leverage machine learning in order to generate structural-phenotypic maps which can be used to generate novel compounds and predict phenotypic data for the compounds.

In some examples, disclosed embodiments of machine learning models may generate a prediction of the effect of a compound based on properties of the compound, such as chemical structure. Such machine learning models can generate a prediction using observational datasets acquired during multiple trials.

The observational data can include subject data (e.g., behavioral data, physiological data, electrophysiological data, or the like) and/or external data (e.g., environmental data, indications of stimuli or rewards presented to the subject, or the like). In some instances, the non-human animal can be a rodent.

The trials can involve administration of different dosages of the compound to one or more non-human animals. In some instances, the trial may be intended to investigate the effect of the compound on the behavior of the animal. In some embodiments, the compound may be a preclinical compound. For example, the trial may be conducted as part of a drug discovery study of the compound, and the trial may be conducted prior to human clinical trials.

The non-human animal can be a control or wild-type animal; an animal model of disease, dysfunction, or injury; or the like. Animal models of disease can include, for example, models of rare disorders, Parkinson's disease, Huntington's disease, Alzheimer's Disease, or other diseases. Animal models of dysfunction can include, for example, models of autism spectrum disorder, epilepsy, depression, anxiety, anhedonia, apathy, cognitive dysfunction, hyperreactivity, or other dysfunctions. Animal models of injury can include, for example, models of central, peripheral, enteric, somatic, or autonomic nervous system dysfunction or injury. Animal models may be obtained using genetic, environmental, pharmacological, or other manipulations.

Consistent with disclosed embodiments, a testing system can include a controlled environment configured with sensors. The testing system can use the sensors to acquire observational data from a non-human animal administered a compound and disposed within the controlled environment. A machine learning model can predict a class of a compound based on the observational data. The compound class can correspond to an effect of the compound when administered to a human.

Consistent with disclosed embodiments, the sensors can include cameras, electrophysiological recording devices (e.g., EEG recording devices, or the like), piezoelectric sensors, infrared detectors, radiofrequency detectors, and the like. In some embodiments, a sensor can include both an emitter such as an infrared beam, a radiofrequency, a source of heat, a source of optical signals, or other such source, as well as a receiver, such as a component configured to receive data or electronic communications. Multiple cameras can be used to acquire depth or 3D information. The particular configuration of sensors can be selected or adapted based on the subject and/or the compound administered.

Consistent with disclosed embodiments, the observational data can include position data, motion data, thermal data, force data, EEG signals, respiration data, or the like. For example, observational data for a mouse can include head, body center, and paw positions (e.g., x, y, z coordinates or the like) and time derivatives thereof (e.g., velocity and acceleration); heart rate; EEG data; eye temperature; and/or other suitable measures. In some embodiments, observational data can be acquired and stored for subsequent processing.

Disclosed embodiments may involve screening compounds using an animal model of disease, dysfunction, or injury. An animal model subject can be treated with a compound. A class for the animal model subject can be predicted. As may be appreciated, absent a therapeutic effect of the compound, the animal model subject may be identified as having the disease, dysfunction, or injury. When the compound has a therapeutic effect that addresses the disease, dysfunction, or injury, the animal model subject may instead be identified as being a vehicle, wild-type animal, or similar control subject. In some embodiments, a likelihood or probability of identifying the animal model subject as being such a control subject can be used to quantify the therapeutic effect of the compound.

In some embodiments, administering a compound to a non-human animal may involve providing a compound to the animal, such as by injecting or adding to the food or water provided to the animal. Consistent with disclosed embodiments, an administration effect can be the predicted response (e.g., biological, physiological, or psychological response) of a human to the administration of the compound. The effect of administering the compound may or may not be therapeutic. In some embodiments, the administration effect of the compound can be described in terms of classes of drugs. In some embodiments, the administration effect of the compound can be described in terms of a drug class and subclass. In some embodiments, a drug class can represent a coarse categorization and a drug subclass can represent a finer categorization. In some embodiments, a drug subclass can be associated with a mechanism of action or a type of chemical structure. In some embodiments, an administrative effect can represent or reflect an overall activity, potency, or side effect(s) of the compound. In some embodiments, an administrative effect can represent or reflect a phenotype of an animal model of disease, dysfunction, or injury (e.g., following administration of a placebo, therapeutically ineffective compound, or the like to such an animal model). In some embodiments, an administrative effect can represent or reflect a normal behavioral phenotype (or a recovery of a normal phenotype in such an animal model following administration of a therapeutic compound). For example, recovery of a normal behavioral phenotype (e.g., indicated by a predicted class of “wild-type,” “vehicle,” “normal,” “control” or the like) in a non-human animal model of a disease. dysfunction, or injury, can indicate that a human having the disease, dysfunction, or injury would therapeutically benefit from administration of the compound. In some embodiments, the administration of the compound may depend on dosage of the compound. Phenotypic signatures may be obtained for a compound corresponding to minimal and maximal therapeutic doses of the compound. For example, phenotypic signatures may be obtained for compounds within a range of minimal to maximal dosage having therapeutic effects. In some embodiments, phenotypic signatures may be obtained for a compound corresponding to minimal and maximal tolerated doses. For example, data may be obtained for doses which avoid producing toxic effects and/or side effects in the non-human animal, or for predicted toxic doses in a human.

As may be appreciated, the disclosed embodiments are not limited to any particular listing of drug classes. In some embodiments, as described herein, a compound may be predicted to belong to (or be associated with) more than one drug class (or subclass). In some embodiments, such classes can include antidepressant, anxiolytic, antipsychotic, cognitive enhancer, hallucinogen, anticonvulsant, mood stabilizer, psychostimulant, or the like. For example, such classes can include antidepressant (e.g., MAO inhibitors, adrenergic and serotonergic receptors antagonists, mixed or specific serotonin and norepinephrine reuptake inhibitors, triple monoamine reuptake inhibitors, tricyclic antidepressants, etc.), analgesic (e.g., opiates, NSAIDs, etc.), anxiolytic (e.g., serotonin 1A receptor agonists. benzodiazepines, mGLUR5 antagonists, neurosteroids, etc.), antipsychotic (e.g., typical, atypical, and other novel antipsychotics), cognitive enhancer/Alzheimer's treatment (e.g., nicotinic cholinergic agonists, cholinesterase inhibitors, phosphodiesterase-4 inhibitors. histamine receptor ¾ antagonists, etc.), anticonvulsant (e.g., GABAb activators, channel blockers, etc.), hallucinogen/psychedelic/treatment-resistant depression treatment (e.g., NMDA antagonist, serotonin 2a receptor agonist, other mixed mechanisms of action, etc.). mood stabilizer (e.g., tricyclic mood stabilizers, lithium, channel blockers, GABA reuptake inhibitors, etc.), psychostimulant and/or ADHD medication (e.g., monoamine releasers, reuptake inhibitors and agonists, mGLURS antagonists, etc.), anesthetic, sedatives, anxiogenic, anticognitive, side effect/toxic, hypnotics (e.g., histamine 1 receptor antagonists, benzodiazepines, adrenergic agonists, etc.), other therapeutic area, inactive, hypnotic, and/or vehicle.

In some embodiments, a listing of drug classes can include an “unknown” class. The unknown class can include compounds that do not fall into the other drug classes (e.g., active compounds having administration effects distinct from the administration effects associated with the other drug classes).

The disclosed embodiments are not limited to any particular listing of drug subclasses. In some embodiments, antidepressants can be further sub-classified as NMDA antagonists, serotonin 2A antagonists and serotonin reuptake inhibitors, Monoamine oxidase A (MAOA) and Monoamine oxidase B (MAOB) inhibitors, norepinephrine and dopamine reuptake inhibitors, selective serotonin reuptake inhibitors, tricyclics, or the like. In some embodiments, analgesics can be further sub-classified as opiate agonists, non-steroidal anti-inflammatories, or the like. In some embodiments, anxiolytics can be further sub-classified as glutamate receptor 5 agonists, serotonin 1 A receptor agonists, benzodiazepines, or the like. In some embodiments, sedatives (or hypnotics) can be further subclassified being z-drugs, or the like. In some embodiments, antipsychotics can be further subclassified as atypical antipsychotics, typical antipsychotics, or the like. In some embodiments, cognitive enhancers can be further subclassified as nicotinic agonists, NMDA antagonists, cholinesterase inhibitors, or the like.

In some embodiments, a compound may be predicted to have a general “activity” level, which may be or reflect the extent to which phenotypic signatures for the compound are distinguishable from phenotypic signatures for a vehicle.

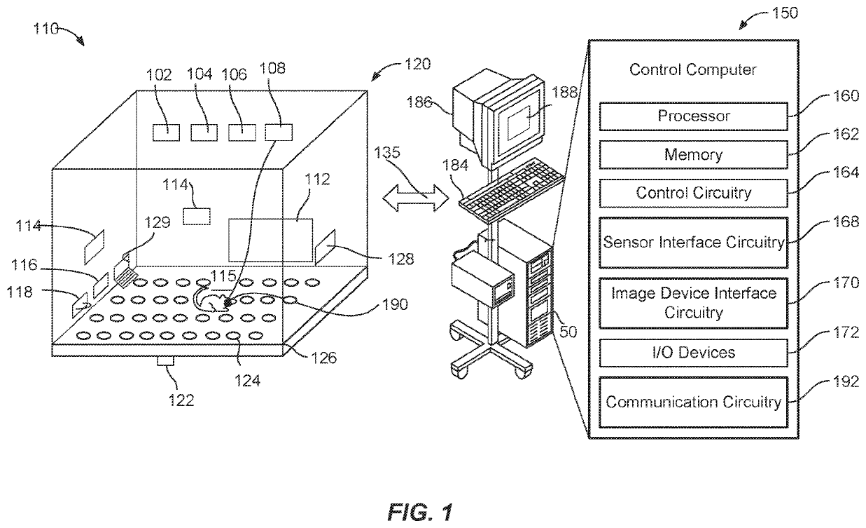

FIG. 1 illustrates a system 110 for acquiring (and optionally analyzing) observational data concerning a non-human animal 115, consistent with disclosed embodiments. System 110 may include a control computer 50 communicating 135 with a computer-controlled enclosure 120. System 110 can include sensors configurable to acquire data during an experiment on a non-human animal 115. System 110 can include a computer-controlled enclosure 120 configured to contain the non-human animal 115 during the experiment. In some embodiments, the computer-controlled enclosure 120 can include actuators configurable to interact with non-human animal 115. For example, sensors and/or actuators may include an aversive stimulus probe 118 that may be deployed and retracted, a motor challenge 126 that may be deployed or retracted so as to force non-human animal 115 to walk on a plurality of physical obstacles 124 arranged in an array with a predefined pitch to provide a motor challenge, lighting 104 that may be configured to change illumination intensity levels and/or light wavelengths as applied to the non-human animal 115, a tactile stimulator 116 to administer tactile stimuli to the non-human animal 115, a top camera 106, a top thermal camera 102, a first side camera 128, a second side camera 114, a floor force sensor 122, waterers and feeders 129, and as additional actuators 22 for applying any additional suitable stimuli to the non-human animal 115 and/or any additional sensors.

In some embodiments, computer-controlled enclosure 120 can include social pod 112 (e.g., shown as an opening in enclosure 120 in FIG. 1 that enables social pod 112 to be affixed to enclosure 120 via the opening). Affixing social pod 112 to computer-controlled enclosure 120 can enable a second non-human animal in social pod 112 to interact with non-human animal 115.

In some embodiments, computer-controlled enclosure 120 can include a 3D camera. A 3D camera can be implemented using multiple 2D cameras configured and arranged around enclosure 120 to obtain 3D image data. In some embodiments, the 3D camera can be implemented using at least one of top camera 106, the first side camera 128, the second side camera 114, or additional cameras.

In some embodiments, the first side camera 128 and the second side camera 114 may be oriented to capture images in the computer-controlled enclosure 120 from different perspectives. An angle between the orientation of the first side camera 128 and the second side camera 114 may be any suitable angle to capture movement throughout the computer-controlled enclosure 120. For example, the angle may be 90 degrees, though other angles may be used (e.g., any angle from about 1 degree to about 179 degrees), such that imagery from both the first side camera 128 and the second side camera 114 may be processed to determine movement within the computer-controlled enclosure 120.

In some embodiments, the plurality of sensors may include sensors associated with some actuators to capture specific responses to the actuators. In some embodiments, the plurality of sensors may be combined with multiple actuators to challenge the test subject to react to various events, which are recorded and analyzed by the system. The resulting ethophysiogram, or collection of physiological and behavioral responses. may create a dataset (e.g., a content-rich dataset) suitable for use with the disclosed systems and methods.

Consistent with disclosed embodiments, the control computer 150 may include at least one processor 160 for executing suitable computer applications as described herein for performing the analysis of the behavior of the non-human animal 115, at least one memory and/or suitable storage device denoted by memory 162 for storing the computer code and any databases used in the analyses of the acquired data over the predetermined time period, control circuitry 164 for controlling the plurality of actuators in accordance with the experimental plan, sensor interface circuitry 168 for outputting data from the plurality of sensors, image device interface circuitry 170 for receiving the output data from any or all of the cameras and/or thermal cameras, input and output (I/O) devices 172, and/or communication circuitry 192 to enable the control computer 150 to communicate over any suitable communication network.

In some embodiments, the I/O devices 172 may include, for example, a display 186 and/or a keyboard 184. Keyboard 184 may allow a user or operator of system 110 to input data to the control computer 150. The at least one processor 160 may control a graphical user interface (GUI) 188 displayed on the display 186. The GUI 188 may display any suitable parameters and/or data visualizations related to the experimental session and/or results of analyses of the data acquired by the plurality of sensors coupled to enclosure 120 in accordance with the experimental plan.

In some embodiments, a head mount 190 may be placed on the subject's head (e.g., skull). The head mount may include at least one electrode to measure brain electrical activity such as electroencephalogram (EEG) signals, for example. In some embodiments, the head mount 190 may also include at least one accelerometer. The signals from the at least one electrode and/or the at least one accelerometer in the head mount 190 may be coupled via wires to circuitry 108 that can relay the signals for processing to the at least one processor 160.

In some embodiments, system 110 can be configured to acquire electrophysiological data, such as pharmaco-EEG (pEEG) data or the like. System 110 can be configured to record actigraphy and quantitative pEEG from one or more brain regions of unanesthetized non-human animals before and after administration of a compound. Such electrophysiological data can be used, consistent with disclosed embodiments, to identify novel compounds that have a desired effect on electrophysiological activity (e.g., pEEG activity).

In some embodiments, system 110 can be configured to phenotype animal models of disease, dysfunction, or injury. As electrophysiological data can yield pharmaco-dynamic signatures specific to pharmacological action, such data can be used to evaluate translational biomarkers and rapidly screen compounds for potential activity at specific pharmacological targets to provide valuable information for guiding the early stages of drug development.

In some embodiments, phenotypic data may include data from an observational platform. For example, phenotypic data may include observational data as well as electroencephalogram (EEG) data. In some embodiments, observational data may include data corresponding to recordings of the subject (e.g., non-human animal 115). For example, observational data may include animal behavior (e.g., rearing, grooming, sniffing, locomotion), animal posture (e.g., elongated posture, flattened posture, round posture), and animal reactions (e.g., startle). Some disclosed embodiments involve extracting, from the observational data, instant behavioral features. In some embodiments, behavioral data can be used in the extraction of “Instant Features” as represented by sets of values taken by the measured variables. Observational data can be obtained from an experimental session for data acquisition and/or analysis performed over a predefined duration, analyses of videos can be done frame by frame, or of other time series done at the highest resolution possible. Instant behavioral features may describe at least one of size, shape, posture, or movement of the animal. For example, a set of instant behavioral features for a mouse at a given point of time within an experimental session can include the set of x, y, z coordinates and time derivatives (e.g., velocity and acceleration), for its head, body center, paw positions, heart rate and eye temperature.

Some disclosed embodiments involve extracting from the observational data for an animal higher order behavioral features describing motifs, sequences, and/or domains of behavioral function of the animal. a motif can be a particular set or sequence of values (e.g., at least two time samples) in a time series stream that occurs with a probability higher than chance. Values can represent instant features and/or states. Transitions between every pair of discrete states can be from one instant feature to another, or also to the same feature. This can be a “first order” motif. In some instances, a set or sequence of several states can occur with a certain probability. Such a set or sequence of “n” states can be labeled an “n-order” motif.

In some embodiments, a motif can be defined a priori and identified in a time series, or a motif can be discovered using unsupervised machine learning methods. As described herein, the time series can include observational data. This observational data can include subject data (e.g., behavioral data, physiological data, or the like) and/or external data (e.g., environmental data, indications of stimuli or rewards presented to the subject, or the like). In some embodiments, a times series can include multiple types of data (e.g., different types of subject data, or a combination of subject data and external data, or the like). In various embodiments, a time series can be specific to a particular type of subject data (e.g., a single kind of behavioral data or physiological data, or the like) or external data.

Consistent with disclosed embodiments. a time series of observational features can be generated. In some embodiments, natural language processing (NLP) can be used for analysis and modeling of such time series data to identify or describe normal, abnormal, and drug-induced changes. In some embodiments, NLP can be used to build a lexicogrammar where the unit of measurement (e.g., features, such as instant features, states, motifs, or domains, or the like) can be understood in the context of a general model that incorporates behavior and drug action occurring at multiple temporal scales and affecting multiple features or combinations of features.

In some embodiments, sensor fusion can be used to identify motifs (e.g., such as the head twitch response), using optical cameras and floor force sensors. For this case, the motif can involve a type of head movement, involving a set or sequence of instant features. A response to an aversive stimulus can trigger a motif that can include an approach to the stimulus, exploration, avoidance, defensive burying response. This can be expected and identified using hardcoded algorithms or supervised machine learning. A motif can be the second highest feature hierarchy below domain.

In some embodiments, a domain can be a particular scenario, area, or type of collected data that can be related to behavioral manifestation, physiological manifestation, or both. A domain can be the highest feature hierarchy. For example, a collection of features representing physiology, such as temperature, can represent a domain. Other domains can be measured by behavioral platforms that can include gait geometry, motor coordination, paw position and paw pressure. Other domains can be exploratory behavior versus consummatory behavior. Domains can be defined using features from the same or across modality such as optical information for overt behavior or thermal information for temperature.

The above hierarchy of features are simply illustrative. Any suitable feature extractor or combination of feature extractors can be employed to form a subject feature profile for a subject (e.g., a test animal) or a group of subjects (e.g., a test animal group). Such a subject or group of subjects can be subject to a modification or can constitute a relevant control group for such a modification. Consistent with disclosed embodiments, the subject feature profile can include any selection or combination of observational features (e.g., instant features. states, motifs, domains, or any combinations thereof). In some embodiments, the subject feature profile can include observational data.

Consistent with disclosed embodiments, analyses can accept as input instant features and/or higher-order features or modified features generated using instant features. Such higher-order features can include states generated using multiple instant features, motifs generated using states (and optionally instant features), and domains generated using motifs (and optionally states and/or instant features). Accordingly, complex phenotypic models can be generated from the instant features. Consistent with disclosed embodiments. such models can support machine-learning-based investigations into the effects of modifications.

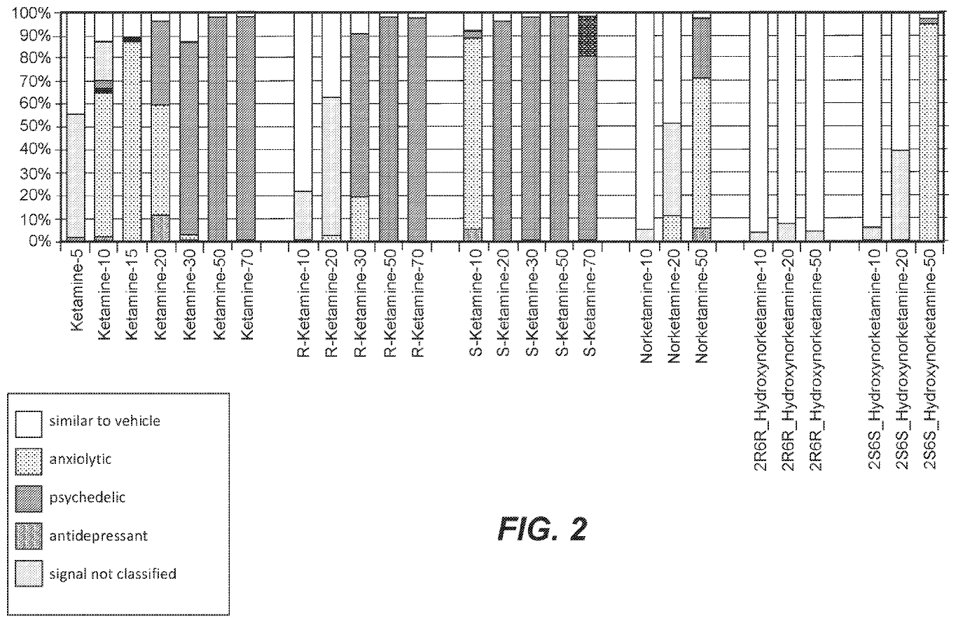

FIG. 2 illustrates a table 200 of exemplary behavioral phenotypic signatures obtained from a system for acquiring observational data, consistent with disclosed embodiments. Obtaining may refer to accessing, acquiring, transmitting, requesting, receiving, or the like. Behavioral phenotypes can be a collection of behavioral features of the animal, observed during the trial or extracted from observations during the trial. Such features can include instant or higher-order features, as described herein. A behavioral phenotype signature can be data generated from the behavioral phenotype of the animal that represents or may be characteristic of the behavioral phenotype. The behavioral phenotype signature can indicate an effect (e.g., treatment effect, side effect, or the like) of the compound administered during the trial (or the behavior characteristics of an animal model of disease, disfunction or injury).

In some embodiments, the behavioral phenotype signature can be a set of therapeutic class probabilities. In some examples, the behavioral phenotypic signature may be an indication of a drug class, such as antidepressant, anxiolytic, antipsychotic, cognitive enhancer, hallucinogen, anticonvulsant, mood stabilizer, psychostimulant, or the like as described herein. As an example, table 200 may illustrate phenotypic signatures obtained from system 110 (as referenced in FIG. 1.) For example, the phenotypic signatures may include instant features and/or higher order behavioral features extracted from observational data obtained from system 110. The phenotypic signatures may correspond to signatures predicted for a non-human animal 115. The predictions in table 200 may include probability distribution generated using a multiclass classifier, such as a classifier that can generate a probability that a particular dose and/or compound belongs to a drug class or subclass (or one or more drug classes). For example, table 200 may illustrate the phenotypic signatures (e.g., obtained from system 110) of ketamine and various metabolites of ketamine as a probability distribution according to the legend.

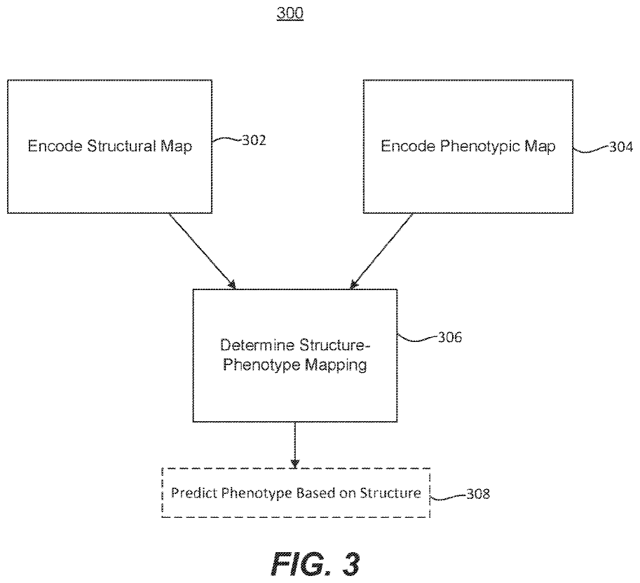

FIG. 3 depicts a block diagram of a method 300 for predicting a behavioral phenotype of a compound using the structure of the compound, consistent with disclosed embodiments. For convenience of description, method 300 may be described herein as being performed by a machine learning system (e.g., such as computer 150, or the like). However, the disclosed embodiments are not so limited. For example, method 300 may additionally or alternatively be performed by another system, such as system 1300. In some embodiments, method 300 may involve configuring a structural-phenotypic model. A structural-phenotypic model may be any model mapping between structural data and phenotypic data (e.g., structural data and phenotypic data associated with an administered compound). In some embodiments, structural data may correspond to the chemical structure of a compound and phenotypic data may correspond to a behavioral phenotypic signature acquired from a non-human animal administered a dose of the compound. In some embodiments, the structural-phenotypic model can be configured to accept structural data and output corresponding predicted phenotypic data. In some embodiments, the structural-phenotypic model may involve one or more machine learning models, such as generative models for predicting phenotypic data based on structural data, and vice-versa.

Method 300 may involve a step 302 of encoding a structural map. Encoding the structural map may include converting (e.g., transforming, translating, or the like) data between formats and/or representational spaces. For example, such encoding may involve projecting input data into a higher or lower dimensional space (e.g., a vector). Encoding a structural map may involve encoding data pertaining to the chemical structure of a compound, such as projecting the structural data to an alternative representational space. In some examples, encoding a structural map may involve encoder-decoder networks. In some embodiments, encoding structural maps may involve determining fingerprints. The fingerprints may be based on structural data from various compounds, and similarity measures and/or distance metrics may be computed for the fingerprints to generate a structural map.

Method 300 may involve a step 304 of encoding a phenotypic map. Encoding a phenotypic map may involve encoding data pertaining to phenotypic behavioral data, such as projecting the phenotypic data to an alternative representational space. Phenotypic data can be obtained from various sources. For example, encoding a phenotypic map may include encoding data obtained from system 110, including instant behavioral features and/or higher order behavioral features extracted from observational data. In some examples, encoding a phenotypic map may involve encoder-decoder networks. In one example, a phenotype encoder may be a neural network trained to output a drug class distribution (e.g., based on reference compounds), and the phenotypical response may be encoded by one or more network layers before classification.

Method 300 may involve a step 306 of determining a structure-phenotype mapping. A mapping may refer to any transformation or projection between representational spaces, such as from one representational space to another. A mapping may be from an n-dimensional space to an m-dimensional space (n and m can be the same or differ, and n and m can be two or more). Determining a structure-phenotype mapping may involve generating a model, such as a network, mapping, or the like, for associating structural and phenotypic data. The structure-phenotype mapping may enable mapping between structural representational spaces and phenotypic representational spaces. In some embodiments, the structure-phenotype mapping may be a machine learning model, such as a generative model. Given a location in the structure representation space, the structure-phenotype mapping can determine a location in the phenotypic representational space. In some embodiments, the structure-phenotype mapping may include a model (or used to train a model) configured to predict minimal and maximal therapeutic doses. For example, a therapeutic dose range may be predicted for a given structure based on the structure-phenotype mapping, including a maximal tolerated dose (e.g., the maximum dose that does not trigger toxic effects or side effects). The structure-phenotype mapping may also be used to determine the type of toxicity of potential compounds. In some embodiments, the structure-phenotype mapping may be used to predict total activity of a compound, which may be the difference in activity from vehicle.

Method 300 may involve a step 308 of predicting phenotype based on structure. Predicting phenotype may refer to predicting a phenotypic behavioral signature, such as predicting a distribution of drug classes to which a given structure may belong. For example, a location in the phenotypic representational space may be predicted based on a location in the structural representational space using the structure-phenotype mapping determined in step 306 (e.g., the machine learning model, or the like). The location in the structural representational space can depend on structural data for the compound. In an example, step 308 may include predicting the similarity of a compound in comparison to a target compound or drug. For example, structural data and/or phenotypic data may be compared between a compound and a target or reference compound.

In some embodiments, given a predicted location in the phenotypic representational space, the phenotypic signature of the compound can be predicted manually, semi-automatically, or automatically. For example, given the predicted location of the compound, a user can identify a similarly located cluster or group of compounds. The predicted behavioral phenotype can be the behavioral phenotype of these similarly located compounds. Likewise, the similarly located compounds can be identified automatically, and the behavioral phenotype of these similarly located compounds identified manually (e.g., through inspection) or automatically. Such identification can include a prediction of behavior, or behavior classification, for the compound when the compound is administered to humans.

In some embodiments, an automated process can be used to identify structures having desired phenotypes, consistent with disclosed embodiments. A machine learning model (e.g., variational autoencoder or similar generative model) can be configured to generate a sample structure. A location in the structural representational space for the structure can be determined, as described herein. The structure-phenotype mapping can be used to determine a predicted location in the phenotype representational space corresponding to the location in the structural representational space. A correction can be determined based on the predicted phenotype location and a desired phenotype location. The correction can be applied to the machine learning model to bias it towards generating structures having desired characteristics.

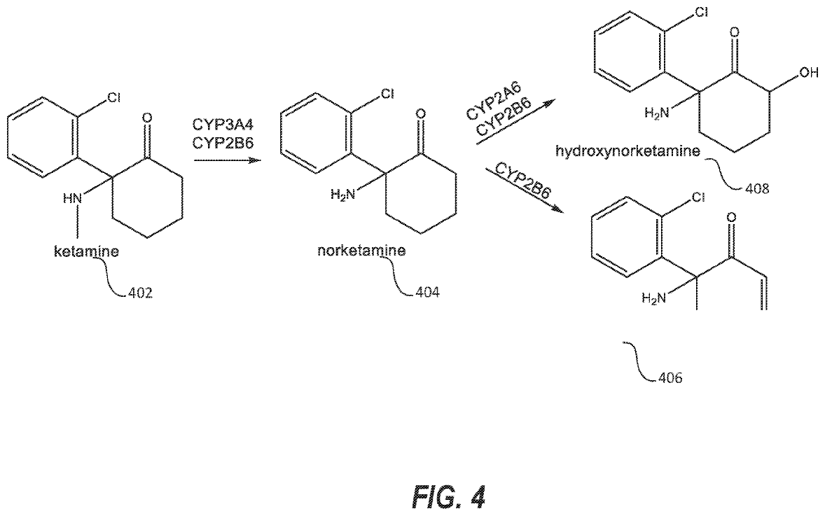

FIG. 4 illustrates exemplary chemical structures, consistent with disclosed embodiments. While chemical structures, such as ketamine 402, norketamine 404, dehydronorketamine 406, and hydroxynorketamine 408, depicted in FIG. 4 may be represented in two-dimensions for ease of illustration, the three-dimensional structural data may capture the compounds' stereochemistry and may also provide information relevant to expected phenotypic signature(s). For example, ketamine 402 may have a chiral center producing two enantiomers, S and R-ketamine, and the metabolites norketamine 404 and hydroxynorketamine 408 may each have two chiral centers. Further, it will be recognized that it may be difficult to predict the potency and similarity of an enantiomer in comparison to a parent compound. For example, referring to FIG. 2, the distributions of table 200 indicate that the racemic mixture and separated enantiomers of ketamine may have a similar anxiolytic signature at lower doses and a psychedelic (e.g., dissociative/hallucinogenic) signature at higher doses. In the example, the S-ketamine enantiomer was more potent than the racemic mixture and more potent than the R-enantiomer. With norketamine as the major metabolite of ketamine, racemic norketamine was less active than ketamine, showing the anxiolytic signature at the doses tested. With hydroxyketamine as a secondary metabolite of ketamine, the 2S6S-hydroxyketamine enantiomer was similar to norketamine, whereas the 2R6R one was inactive at the doses tested. These metabolites may be formed rapidly, with norketamine quantities equating to 80% of the parent drug quantity. Norketamine and ketamine may also be rapidly converted into hydroxynorketamine and other minor metabolites. As such, the potency and signature of the parent compounds cannot be explained by any of the metabolites tested. Thus, given the effects of the changes in 3D conformation (e.g., as demonstrated by comparing 2S6S versus 2R6R-hydroxynorketamine in table 200 as referenced in FIG. 2), it will be appreciated that a model including three-dimensional structural information may be preferable over a model solely based on two-dimensional structural information for predicting behavioral signatures.

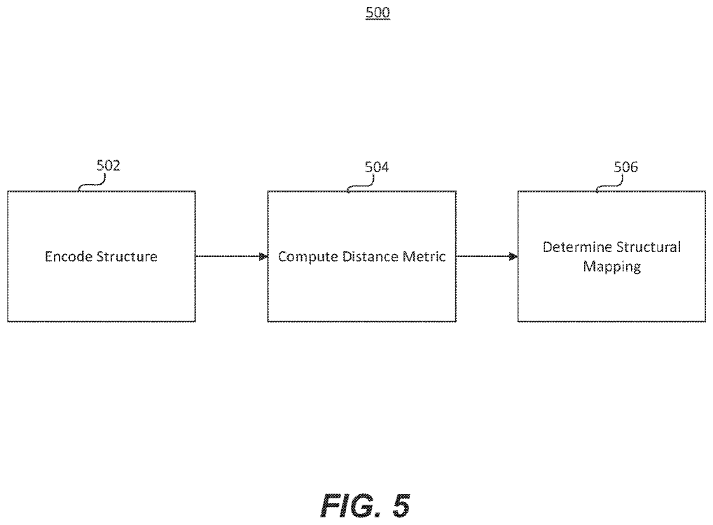

FIG. 5 depicts an exemplary process 500 for mapping structural data to a structure representation space, consistent with disclosed embodiments. Structural data may include any information referring to the arrangement, composition, and/or appearance of compounds and/or molecules. Structural data may include information corresponding to the n-dimensional chemical structure of a compound, including the spatial arrangement of atoms and connectivity, such as chemical bonds (e.g., crystalline structure). Structural data may also include information corresponding to chirality of a compound, such as data describing enantiomers (e.g., racemic mixture composition) or other stereoisomeric data. For example, compounds may contain one or more chiral centers (e.g., a spatial arrangement of atoms about a common center such that there exists a non-superimposable mirror image of such arrangement). Such different stereoisomers may have different therapeutic effects, or none at all. In some embodiments, structural data may include three-dimensional structural data. For example, three-dimensional structural data may include chemical structure models. Three-dimensional structure models may represent three-dimensional structural data, such as information describing the configuration of a compound, including spatial orientation (including preferred spatial orientations) and molecular geometry data, such as bond types and lengths, bond angles, and torsional angles. For example, three-dimensional structural data may involve data describing various conformations of compounds, such as various topologies of molecules (e.g., tertiary topologies of a molecule). In some embodiments, structural data may include two-dimensional structural data. For example, wo-dimensional structural data may include two-dimensional chemical structure models. Two-dimensional structural models may represent two-dimensional structural data, which may refer to the arrangements and connections of atoms within and/or between molecules. For example, two-dimensional data may include the number of atoms and types of atoms in a compound In some embodiments, structural data may include sub-structural data. For example, it may be desired to generate structural mappings based on a common structural motif (e.g., create a model based on a class of compounds rather than an individual compound) Sub-structural data may refer to data pertaining to portions or features of a chemical structure, rather than an entire chemical structure. For example, benzodiazepines may share a common structural motif, such as a fusion of a benzene and diazepine rings (e.g., steroids may share four fused rings as their core with different functional groups attached to the core). In some embodiments, structure-activity maps be generated for the relationships for these substructural motifs rather than the full structures of the individual compounds. In some embodiments, structural data may include electrostatic data, such as attractive and/or repulsive interactions between molecules. For example, electrostatic data may indicate the degree of polarization (e.g., charge distribution) of the bond or the ionization state of a particular functional group. For example, in case of isonitriles (e.g., R—N≡C), the nitrogen atom can carry a partial positive charge. Certain functional groups may be present in an ionized form under particular conditions (e.g., R—NH3+, R—CO2—), which may provide information about how these molecules can interact with other substrates. In some embodiments, structural data may include stereochemistry data. For example, the exact chirality (e.g., the spatial arrangement) for some (or all) of the chiral centers in the molecule may be unknown, and stereochemistry data may indicate the chiral centers for which the exact spatial arrangements may be known and/or unknown. For example, a drug such as propoxyphene may have 2 chiral centers. The exact stereoisomer used in a particular test may be unknown (or unreported), or a mixture of stereoisomers may have been used in the particular test. In some examples, stereochemistry data, which may include sparse stereochemistry data, may be included (e.g., in a mapping) as an indication that the stereochemistry was not known. It will be also recognized that chemical structural data may be readily available. For example, two-dimensional and three-dimensional structural data may be accessible in public datasets, such as through online databases and journals.

In some embodiments, structural data may also include graphical information. For example, in graph networks, the atoms of a chemical structure may correspond to nodes, and the connections between atoms may correspond to links. Another example of structural data may involve molecular fingerprints, which can include characteristic patterns of chemical properties and/or features. In some fingerprints, the structure of a compound may be represented by sets of binary bit strings corresponding to various structural features (e.g., for a given feature of a molecule, the bit value may be 1 if the molecule has the feature or zero if the molecule does not have the feature). For example, Molecular Access System keys (MACCS) may be utilized to encode structural data. In another example, hashed fingerprints may be used to represent the structure of a compound. In some examples of spatial structural data, coordinates of atoms can represent spatial arrangements within a molecule (e.g., using cartesian coordinates of atoms in a three-dimensional space). It will be recognized that the proper representation of the structure of a compound may be important, as minor changes in the structure of a compound can lead to changes in the compound's function.

In some embodiments, process 500 may include a step 502 of encoding structure. Encoding structure may involve encoding structural data into forms which a computer, processor, machine learning model, or the like, can use. For example, machine learning models for encoding structure may include autoencoders, variational autoencoders, recurrent neural networks, generative adversarial networks, or the like. The encoded structural data can be represented by data structures such as arrays, matrices, vectors, or the like.

In some embodiments, process 500 may include a step 504 of computing a distance metric. As may be appreciated, a distance metric can map from two elements of a set to a real number greater than or equal to zero. Here, the distance metric may be any suitable measure of similarity between items of encoded structural data for different compounds (or different doses of the same compound). The distance metric may be computed over various dimensions of the encoded structural data. As may be appreciated, the encoded structural data can be very high dimension data (e.g., tens to thousands of dimensions).

In some embodiments, Tanimoto scores (e.g., Tanimoto distance, similarity or coefficient) can be used as the distance metric. Tanimoto scores may be calculated for a pair of compounds A and B, and the standard Tanimoto distance may be based on the number of common bits of their fingerprints (FA and FB). Tanimoto distance ranges from 0 to 1, as may be defined as T(A,B)=(|F_{A}∩F_{B}|)/(|F_{A}+F_{B}|−|F_{A}∩F_{B}). where ∩ denotes the intersection of the two sets of binary descriptors. A score of 0 may represent no common bits in the fingerprint and a score of 1 indicates the fingerprints may be identical.

In some examples, pairwise Tanimoto scores may be calculated for MACCS two-dimensional fingerprints as an exemplary representation. Tanimoto scores can also be extended to three-dimensional structural encodings. In another example, the Jaccard index may be used as a distance metric.

In some embodiments, process 500 may include a step 506 of determining a structural mapping. The structural mapping may involve projecting the encoded structural data into a compressed feature space (e.g., a latent space). For example, the encoded structural data may be mapped to a latent variable model with axes corresponding to data features. Some disclosed embodiments may involve dimensionality reduction techniques. In such embodiments, the encoded structural data may be higher dimension than the compressed feature space. Such dimensionality reduction can improve the ability of the structure-phenotype model to generalize and can support improved data analysis and visualization. Data points represented closer together in a space may indicate that the data points have more similar features, and data points spaced farther apart may indicate less similarity. In the example of a two-dimensional space, compounds can be assigned coordinates in the space, and structural similarity may be indicated by compounds that are closer together In some embodiments, a structural dataset may be generated based on the mapped structural data in the representation space. For example, the structural dataset may be a data structure (e.g., matrix, array, vector, or the like) including the structural positions of compounds mapped in the representational space.

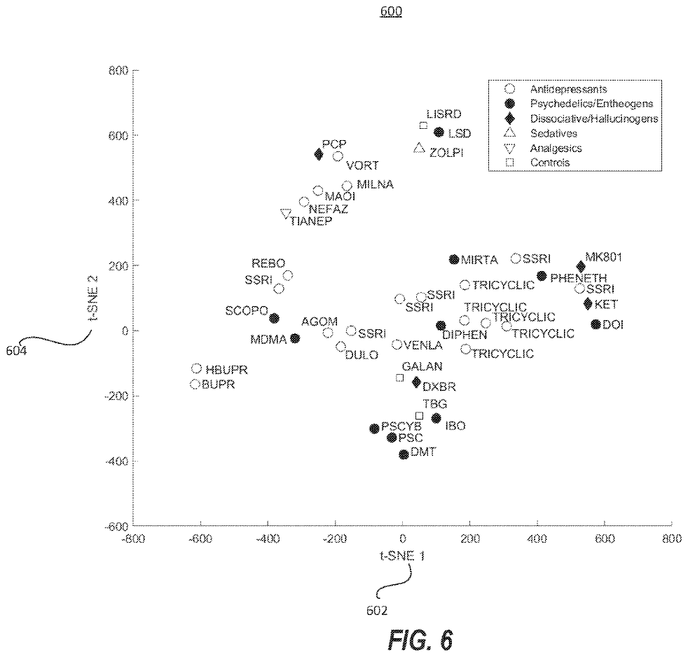

FIG. 6 depicts locations of exemplary compounds in a structure representation space, consistent with disclosed embodiments. Graph 600 may illustrate a reduced-dimensionality representation of a structural mapping. Process 500 may be an exemplary method to generate the representation illustrated in graph 600. For example, graph 600 may illustrate a representation space of structural data mapped with t-distributed Stochastic Neighbor Embedding (t-SNE). The underlying variables (e.g., features) in the space may be represented as axes of the mapping, such as first major t-SNE axis 602 and second major t-SNE axis 604, in the example of two-dimensional graph 600. The structure representation space depicted in graph 600 was generated in accordance with process 500 using MACCS 166-bit fingerprints as encoded structural data, Tanimoto scores as a distance metric, and unsupervised t-SNE for dimensionality reduction. As such, each compound may be assigned a structural position SP, and the structural position of a given compound C may be SP_{C}={t-SNE1(C), t-SNE2(C)}. In some embodiments, data in a representational space may be clustered (e.g., classified). In the example illustrated in graph 600, compounds having known therapeutic effects in humans have been clustered and classified (e.g., as depicted in the legend).

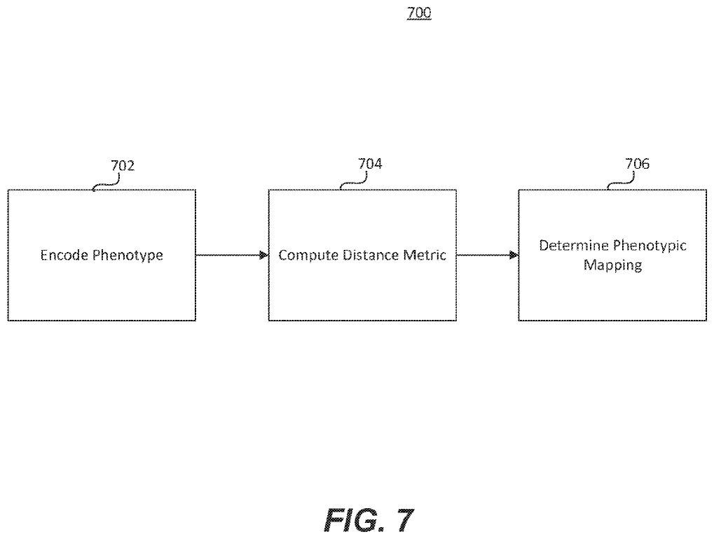

FIG. 7 depicts an exemplary process 700 for mapping phenotypic data to a behavioral phenotype representation space. In some embodiments, process 700 may include a step 702 of encoding phenotype. Encoding phenotype can include acquiring raw data during a trial, or acquiring such raw data and further processing the raw data into various behavioral features. For example, raw data may be obtained from an observational platform (e.g., system 110) and encoded. In the example, the raw data may include over 500,000 observations per sample. In another example, processed data, such as data that has been analyzed, clustered, classified. and/or dimensionally-reduced, may be encoded. Processed data may include summary-level features, such as instant behavioral feature data and/or higher order behavioral data. In the example of processed data, dimensionally-reduced data obtained from system 110 may include over 2000 features per sample. Processed data may also include fingerprints of phenotypic data.

In some embodiments, process 700 may include a step 704 of computing a distance metric. As may be appreciated, the distance metric can map from two encoded phenotypes to a real number greater than or equal to zero. Here, the distance metric may be any suitable measure of similarity between items of encoded phenotypic data for different compounds (or different doses of the same compound). The distance metric may be computed over various dimensions of the encoded phenotypic data. As may be appreciated, the encoded phenotypic data can be very high dimension data (e.g., tens to thousands of dimensions).

In some embodiments, a distance metric can depend on similarities computed for different dosage levels of two drugs. For example, the pairwise similarity S for two drugs A and B at a dose level Aᵢ and Bᵢ may be Sᵢⱼ=S(Aᵢ, Bⱼ), resulting in a N×M matrix, where N and M are the number of doses analyzed for each compound. An overall distance between drugs A and B can then be the maximum similarity at any drug-dose comparison (e.g., S_{AB}=Max(S(Aᵢ, Bⱼ) over all doses i, j).

In some embodiments, the distance metric can be computed using a machine learning model. For example, given a first dataset of encoded phenotypes for a first compound, and a second dataset of encoded phenotypes for a second compound, a binary two-class classifier can be trained to distinguish between phenotypes for the first compound and phenotypes for a second compound. The classifier can be trained using a training portion of the two datasets and the performance of the classifier can be evaluated using a holdout portion of the two datasets. The performance of the classifier on the holdout portion can be used as a distance between the first compound and the second compound. For example, the classifier may generate a shortest distance (e.g., maximal similarity) between two compounds when the holdout samples are classified at random (e.g., 50%, which can be rescaled to 0 distance). When the holdout samples are perfectly classified, then separation between the datasets may be maximal (e.g., 100%, which can be rescaled to a distance of 1). The distance metric may also be extracted from the probability that the classifier results are due to chance (e.g., using a permutation test). Using binary classifiers to measure the distance between two phenotypic datasets may provide the advantage of being agnostic to the type of phenotypic signature the drug or compound may have (e.g., antidepressant, antipsychotic, etc).

In some embodiments, process 700 may include a step 706 of determining a phenotypic mapping. A phenotypic mapping may be a projection of phenotypic data onto a representational space. For example, encoded phenotypic data and/or distance metrics computed for the phenotypic data may be mapped to a representational space, such as a latent variable model space. The variables (e.g., features) represented in the map may be the major t-SNE axes for the map. Using a computed distance metric, compounds can be assigned positions in the space via coordinate sets. For example, S_{AB}=Max(S(Aᵢ, Bⱼ)) can be used to assign a pairwise distance. As such, the phenotypic position for a drug C in the mapped space may be PP_{C}={t-SNE1′(C), t-SNE2′(C)}. In some embodiments, a phenotypic machine learning model may be used to map phenotypic data to a representation space. The phenotypic machine learning model may be a classifier, as described herein. The phenotypic machine learning model may also be an encoder configured for dimensional reduction. In some embodiments, a phenotypic dataset may be generated based on the mapped phenotypic data in the representational space. For example, the phenotypic dataset may include the coordinates for the compounds in the space. In some examples, the phenotypic dataset may include the set of phenotypic positions for the compounds.

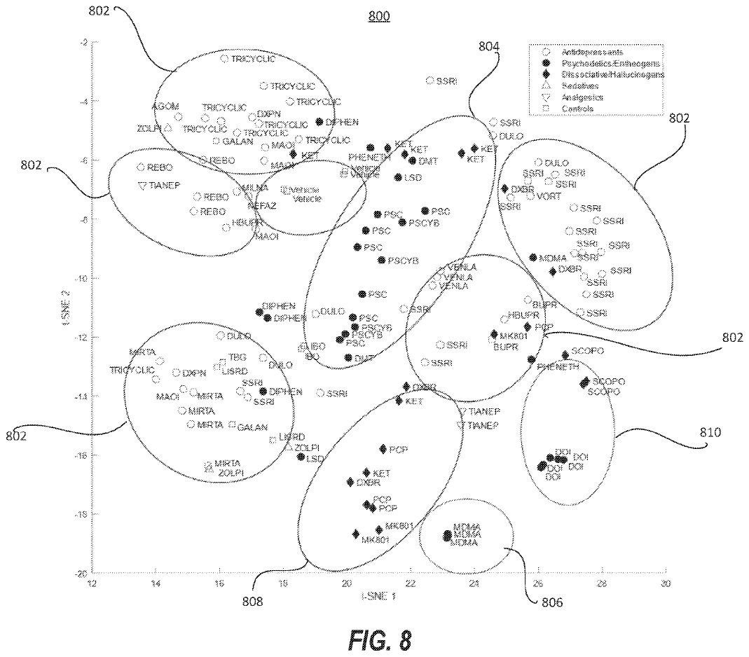

FIG. 8 depicts locations of exemplary compounds in a behavioral phenotype representation space, consistent with disclosed embodiments. Each point in graph 800 can correspond to one or more trials in which an animal (e.g., a mouse, or the like) was administered a particular dose of a particular drug. As may be appreciated, process 700 can be used to generate representations similar to the representation illustrated in graph 800. For example, graph 800 may illustrate a method of mapping phenotypic data as described in step 706. The t-SNE method may be applied to a distance metric (e.g., pairwise similarity) to identify clusters of drugs with similar behavioral phenotype signatures. In another example, a phenotypic machine learning model may identify the clusters of drugs. In the example illustrated in graph 800, cluster(s) 802 may include compounds identified as antidepressants, cluster 804 as psychedelics, cluster 806 as compounds identified as psychedelics/entheogens with antidepressant potential (e.g., MDMA), cluster 808 as compounds which block NMDA receptors, and cluster 810 as Dimethoxy-4-iodoamphetamine (DOI). As described herein, the clusters in graph 800 may be generated from observational data in an unsupervised manner, as it may not be necessary to know the a priori therapeutic classes each compound belongs to. In some embodiments, the space the phenotypic data is mapped to may be different than the space structural data is mapped to. It will be recognized that, in comparison with graph 800 of FIG. 8, clusters based on the positions of phenotypic data may not be the same as the groupings in graph 600 of FIG. 6 corresponding to structural data. As such, it will be appreciated that distance metrics and representation in respective phenotypic and structural spaces may be different in the different mappings.

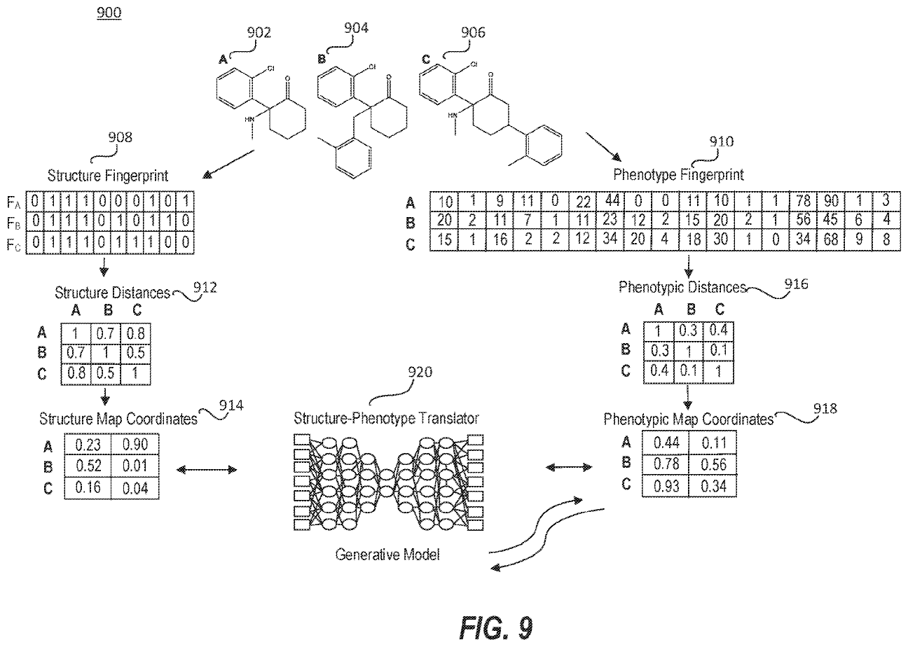

FIG. 9 depicts a process 900 for generating a structural-phenotypic mapping using distances, consistent with disclosed embodiments. Process 900 may include obtaining data corresponding to compounds from a set of compounds administered to non-human animals, including compound A 902, compound B 904, and compound C 906. The data may include structural data corresponding to the compounds as well as observational data. For example, the observational data may be obtained from system 110. Process 900 may include a step 908 of encoding the structural data. The structural data may be encoded in step 908 into a structure fingerprint, such as MACCS fingerprint (e.g., SP.). Process 900 may also include a step 910 of encoding phenotypic data. For example, the observational data may be encoded by similarity analysis (e.g., with a two-class classifier) to a phenotypic fingerprint.

Process 900 may include a step 912 of determining structural distances. The distances may be determined between the compounds in the sets of compounds (e.g., pairwise). For example, Tanimoto scores may be computed between structural data corresponding to compound A 902 and structural data corresponding to compound B 906, and similarly between compounds B and C, and A and C. The structural data may then be mapped to a location in a representation space in step 914. For example, the structural distance data in step 912 may be mapped to a representation space for structure using dimensionality reduction methods described herein, including the t-SNE method. Step 914 may also include generating structural datasets based on the location of compounds in the representation space. For example, a structural dataset may include the coordinates of compound A 902, compound B 904, and compound C 906 in the representation space. In this example, the representation space is two-dimensional (e.g., two coordinates for each compound), but the disclosed embodiments are not so limited, and may include higher-dimensional representation spaces (e.g., 3, 4, 5, or higher-dimensional spaces). As may be appreciated, the representation space can be lower-dimension than the original encoded structural data.

Process 900 may include a step 916 of determining phenotypic distances. The distances may be determined between observational data for compounds in the sets of compounds (e.g., pairwise). For example, a similarity distance or binary classifier output may be computed between observational data corresponding to compound A 902 and observational data corresponding to compound B 906, and similarly between compounds B and C, and A and C (e.g., PP_{C}). The phenotypic data may then be mapped to a location in a representation space in step 918. For example. the phenotypic distance data in step 916 may be mapped to a representation space for phenotype using dimensionality reduction methods described herein, including the t-SNE method. Step 918 may also include generating phenotypic datasets based on the location of compounds in the representation space. For example, a phenotypic dataset may include the coordinates of compound A 902, compound B 904, and compound C 906 in the representation space. In this example, the representation space is two-dimensional (e.g., two coordinates for each compound), but the disclosed embodiments are not so limited, and may include higher-dimensional representation spaces (e.g., 3, 4, 5, or higher-dimensional spaces). As may be appreciated, the phenotypic representation space can be lower-dimension than the original encoded phenotypic data.

Process 900 may include a step 920 of generating a structure-phenotype map. For example, the structure-phenotype map may be a mapping between the structural dataset and phenotypic dataset generated in steps 914 and 918. The structure-phenotype map may enable prediction of a location in the phenotypic representation space, given a location in the structural representation space, thereby associating the structural coordinates SP_{C} and phenotypic coordinates PPₒ.

In some embodiments, the structure-phenotype map may be generated with the phenotypic dataset and the structural dataset using neural networks, such as encoder-decoder models. The mapping may allow translation from structure to phenotype (and in some embodiments, from phenotype to structure).

In some embodiments, the structure-phenotype map may be used to configure a machine learning model, such as to train a machine learning model. The structure-phenotype map may be used to train a generative machine learning model. The model may capture the relationships between structures, phenotypes, and between structure-phenotypes mappings, enabling the generation of novel compounds that can be expected to produce a desired phenotype. For example, given a novel compound, the generative model can be trained to predict the phenotypic signature (e.g., the novel compound's position in a phenotypic map). Based on the result of the position in the phenotypic map, if the phenotypic signature is undesired, such as a phenotypic signature that has no indication (e.g., far away from other positions in the map) or has an unintended indication (e.g., associating with a different cluster than intended), the generative model can be updated and reconfigured. As such, the generative model can be configured to map between encoded phenotypic data and encoded structural data to predict phenotypic data for novel compounds, enabling de novo drug discovery. In an example, given a structural map coordinate for a compound, the mapping may be used to predict the corresponding phenotypic map coordinate for the compound. As such, by modifying the compound and analyzing the effects of the structural change on the structural fingerprint and the structural distance scores, the mapping may be used to determine the phenotypic signature of the modified compound based on the modified structural map coordinate. In some embodiments, a therapeutic class may be predicted based on predicted phenotypic data. For example, the generative model, or a separate machine learning model, may be configured to generate a prediction of one or more therapeutic classes for a novel compound based on the predicted phenotypic data, such as phenotypic structural data. In some embodiments, the generative model may be configured to predict minimal and maximal therapeutic doses (e.g.,, a therapeutic dose range). In an example, the generative model may be able to predict the similarity between a novel or modified compound and a target compound or drug. In the example, the generative model may use similarity measures described herein, such as distance metrics.

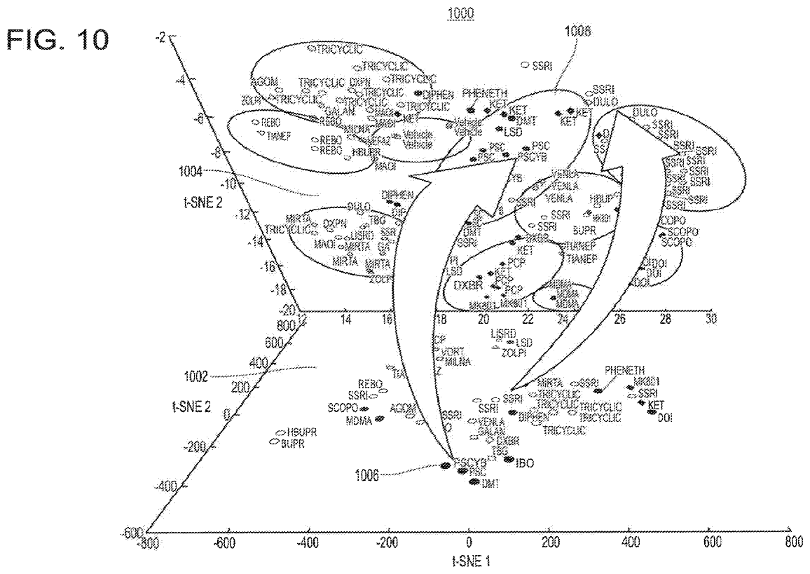

FIG. 10 depicts an exemplary mapping 1000 between a structural representation space and a behavioral phenotype representation space, consistent with disclosed embodiments. Mapping 1000 may be an example of a structure-phenotype mapping generated in step 920 as referenced in FIG. 9. The mapping 1000 may be between exemplary structural representation space 1002 and phenotypic representation space 1004. For example, structural data of compound psilocin 1006 may map to a first cluster 1008 of phenotypic data. Based on the mapping 1000, a machine learning model can be configured to generate new compounds that map onto a desired phenotypic cluster in the phenotypic representation space 1004.

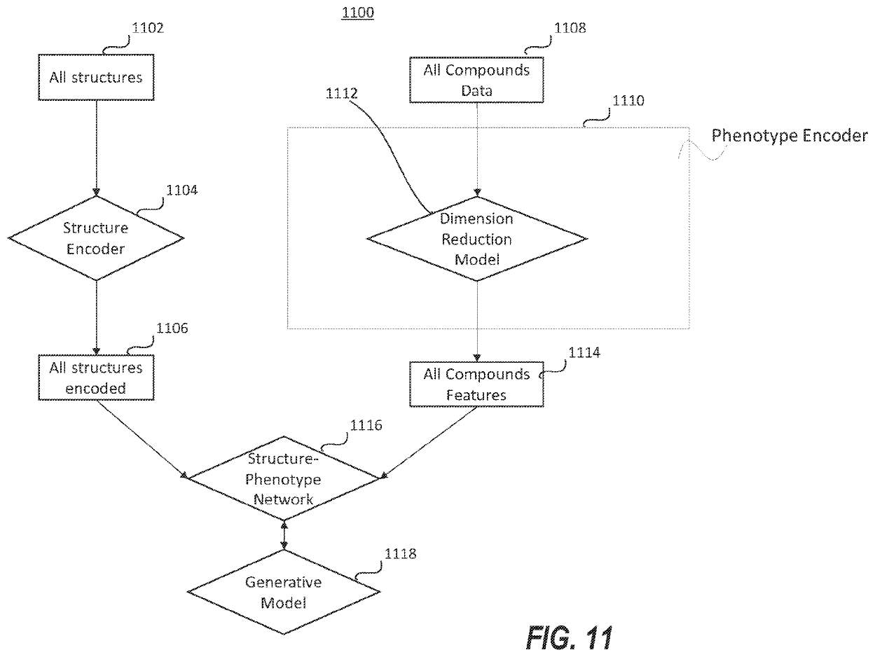

FIG. 11 depicts a process 1100 for generating novel compounds based on structural and phenotypic data, consistent with disclosed embodiments. Process 1100 may include a step 1102 of obtaining structures for compounds in an administered set of compounds. Process 1100 may use structure encoder 1104 to encode the structures to generate a structural dataset 1106 of encoded structures. For example, structural encoder 1104 may encode structure into a format configured as data structure (e.g., a vector or array). In one example, a system such as a Simplified molecular-input line-entry system (SMILES) may encode structural data into a string of symbols (e.g., ASCII strings). In another example, structural encoder 1104 may include image encoders, image embeddings, contrastive learning, and/or cosine similarities to encode structural data (e.g., Contrastive Learning and leave-One-Out-boost for Molecule Encoders).

Process 1100 may also include a step 1108 of obtaining phenotypic data corresponding to the compounds in the administered set of compounds. In some examples, the phenotypic data may include observational data from an observational platform (e.g., system 110). For example, raw data captured by videos and sensors of an observational platform may be processed to extract features. The phenotypic data, including the raw data and/or extracted features as described herein, may be inputs to a phenotype encoder 1110. In some examples, phenotype encoder 1110 may include a dimensionality reduction model 1112. The dimensionality reduction model 1112 may include any method of reducing the dimensionality of the input data. For example, the dimensionality reduction model 1112 may take raw data including pixel-level data from the cameras and sensors, and extract phenotypic features 1114 from the raw data, including instant behavioral features (e.g., features which can vary frame by frame, such as animal body part position, distance traveled) as well as higher-order behavioral features (e.g., motifs, sequences, domains, and/or transitions between animal behaviors). The extracted features may also include summarized data, such as binned data, mean data, and/or standard deviation data.

Consistent with disclosed embodiments, the dimensionality reduction model 1112 can generate a phenotypic dataset 1114 of encoded phenotypes. The phenotypic dataset 1114 can include entries for all compounds in the set of administered compounds. Using the structural dataset 1106 and the phenotypic dataset 1114, the structure-phenotype map 1116 may be generated. In some embodiments, a generative model 1118 may be configured using the structure-phenotype map 1116. For example, the generative model may be trained using structure-phenotype map 1116, as described herein, to generate structures predicted to induce, when administered, desired behavioral phenotypes.

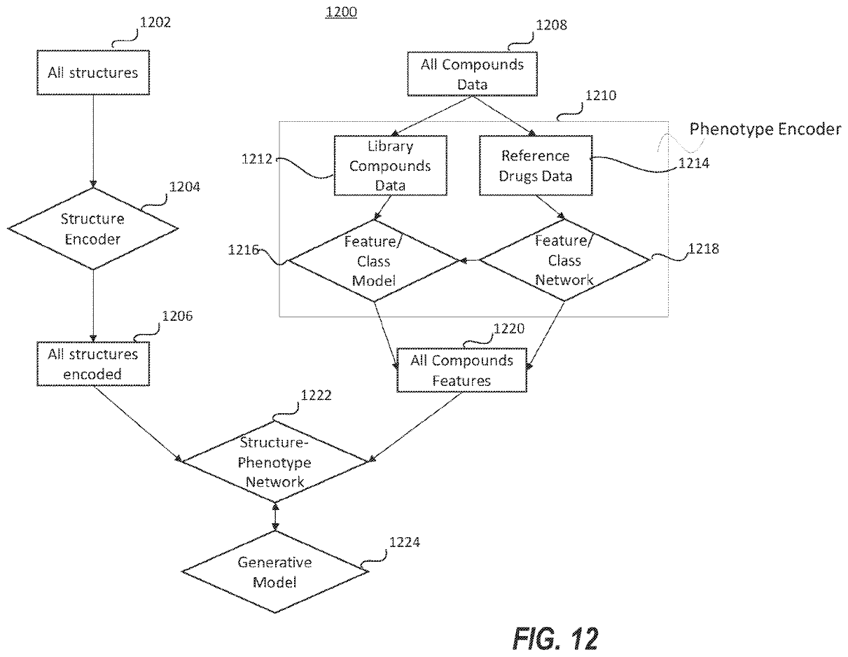

FIG. 12 depicts a process 1200 for generating novel compounds based on structural and phenotypic data, consistent with disclosed embodiments. Process 1200 may include a step 1202 of obtaining structures for compounds in an administered set of compounds. Process 1200 may use structure encoder 1204 to encode the structures to generate a structural dataset 1206 of encoded structures. For example, structure encoder 1204 may be an autoencoder, variational autoencoder, image encoder, contrastive learning system, or the like.

Process 1200 may also include a step 1208 of obtaining phenotypic data corresponding to the compounds in the administered set of compounds. In some examples, the phenotypic data may include observational data corresponding to novel compounds and reference compounds. In some embodiments, process 1200 may include generating a phenotypic signature prediction (e.g., a therapeutic class prediction). Some disclosed embodiments may involve obtaining class labels for a subset of the set of compounds (e.g., reference compounds) and training a phenotypic machine learning model using the class labels. In some examples, the reference compounds may be compounds having known behavioral responses. For example, a compound such as diphenhydramine may be a reference compound with a known phenotypic signature (e.g., class label) of depressive potential when administered to a human. The phenotypic machine learning model may be a phenotype encoder 1210 which may encode the observational data 1208. In some embodiments, observational data 1208 may include data corresponding to reference compounds 1214. In some examples, the class labels may be included in a feature-class network 1218. Phenotype encoder 1210 may also encode novel compounds. For example, novel compounds in the observational data 1208 may be an input to compounds library 1212. Based on the reference compounds 1214 and known class labels in feature-class network 1218, a feature class model 1216 may generate class predictions for the novel compounds. In some embodiments, the phenotypic signatures from the reference compounds, or a control compound may be used to normalize the phenotypic signature. Based on the phenotype encoder 1210, a phenotypic dataset 1220 may be generated for all compounds, including reference and novel compounds. Using the structural dataset 1206 and the phenotypic dataset 1220, the structure-phenotype map 1222 may be generated. In some embodiments, a generative model 1224 may be configured using the structure-phenotype map 1222. For example, the generative model may be trained using structure-phenotype map 1222, as described herein, to generate structures predicted to induce, when administered, desired behavioral phenotypes.

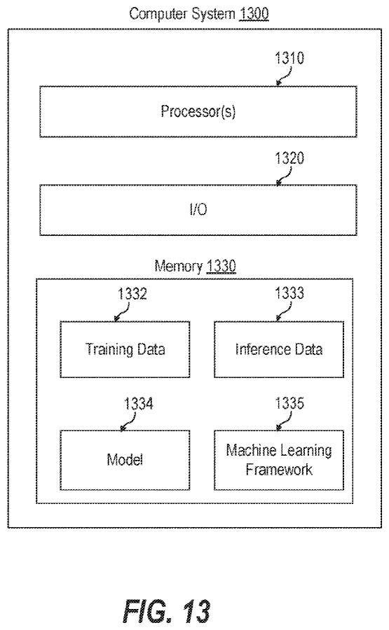

FIG. 13 depicts an exemplary computing system 1300 suitable for generating machine learning models, consistent with disclosed embodiments. In some embodiments, machine learning systems described herein can be implemented using computing system 1300. Similarly, one or more methods and processes described herein, including methods 300, 500, 700, 900, 1100, and 1200, can be implemented using computing system 1300. As will be appreciated by one skilled in the art, the components and arrangement of components included in computing system 1300 may vary. For example, as compared to the depiction in FIG. 13, computing system 1300 may include a larger or smaller number of processors, I/O devices, or memory units. In addition, computing system 1300 may further include other components or devices not depicted that perform or assist in the performance of one or more processes consistent with the disclosed embodiments. The components and arrangements shown in FIG. 13 are not intended to limit the disclosed embodiments, as the components used to implement the disclosed processes and features may vary.

Processor 1310 may be or include known computing processors, including a microprocessor. Processor 1310 may be or include a single-core or multiple-core processor that executes parallel processes simultaneously. For example, processor 1310 may be a single-core processor configured with virtual processing technologies. In some embodiments, processor 1310 may use logical processors to simultaneously execute and control multiple processes. Processor 1310 may implement virtual machine technologies, or other known technologies to provide the ability to execute, control, run, manipulate, store, etc., multiple software processes, applications, programs, etc. In another embodiment, processor 1310 may include a multiple-core processor arrangement (e.g., dual core, quad core, etc.) configured to provide parallel processing functionalities to allow execution of multiple processes simultaneously. One of ordinary skill in the art would understand that other types of processor arrangements could be implemented that provide for the capabilities disclosed herein. The disclosed embodiments are not limited to any type of processor, Processor 1310 may execute various instructions stored in memory 1330 to perform various functions of the disclosed embodiments described in greater detail below. Processor 1310 may be configured to execute functions written in one or more known programming languages.

Computer program code for carrying out operations (e.g., operations consistent with disclosed embodiments) may be written in any suitable programming language (or combination of two or more programming languages), including object-oriented programming languages such as Java, Smalltalk, C⁺⁺ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

I/O device 1320 may include at least one of a display, an LED, a router, a touchscreen, a keyboard, a microphone, a speaker, a haptic device, a camera, a button, a dial, a switch, a knob, a transceiver, an input device, an output device, or another I/O device to perform methods of the disclosed embodiments.

I/O device 1320 may be configured to manage interactions between computing system 1300 and other systems using a network. In some aspects, I/O device 1320 may be configured to publish data received from other databases or systems not shown. This data may be published in a publication and subscription framework (e.g., using APACHE KAFKA), through a network socket, in response to queries from other systems, or using other known methods. In various aspects, I/O 1320 may be configured to provide data or instructions received from other systems. For example, I/O 1320 may be configured to receive instructions for generating data models (e.g., type of data model, data model parameters, training data indicators, training parameters, or the like) from another system and provide this information to machine learning framework 1335. As an additional example, I/O 1320 may be configured to receive data from another system (e.g., in a file, a message in a publication and subscription framework, a network socket, or the like) and provide that data to programs or store that data in, for example, data set 1332 or model 1334.

In some embodiments, I/O 1320 may include a user interface configured to receive user inputs and provide data to a user (e.g., a data manager). For example, I/O 1320 may include a display, a microphone, a speaker, a keyboard, a mouse, a track pad, a button, a dial, a knob, a printer, a light, an LED, a haptic feedback device, a touchscreen and/or other input or output devices.

Memory 1330 may be a volatile or non-volatile, magnetic, semiconductor, optical, removable, non-removable, or other type of storage device or tangible (i.e., non-transitory) computer-readable medium, consistent with disclosed embodiments. As shown, memory 1330 may can be configured to store inference data 1333 and training data set 1332. Inference data 1333 or training data set 1332 may be encrypted or unencrypted. Memory 1330 may also include models 1334, including weights and parameters of neural network models or other machine learning models.

Machine learning framework 1335 may include one or more programs (e.g., modules, code, scripts, or functions) used to perform methods consistent with disclosed embodiments. Programs may include operating systems (not shown) that perform known operating system functions when executed by one or more processors. Disclosed embodiments may operate and function with computer systems running any type of operating system. Machine learning framework 1335 may be written in one or more programming or scripting languages. One or more of such software sections or modules of memory 1330 may be integrated into a computer system 1300, non-transitory computer-readable media, or existing communications software. Machine learning framework 1335 may also be implemented or replicated as firmware or circuit logic.

Machine learning framework 1335 may include programs (scripts, functions, algorithms) to assist creation of, train, implement, store, receive, retrieve, and/or transmit one or more machine learning models. Machine learning framework 1335 may be configured to assist creation of, train, implement, store, receive, retrieve, and/or transmit, one or more ensemble machine learning models (e.g., machine learning models comprised of a plurality of machine learning models). In some embodiments, training of a model may terminate when a training criterion is satisfied. Training criteria may include the number of epochs, training time, performance metric values (e.g., an estimate of accuracy in reproducing test data), or the like. Machine learning framework 1335 may be configured to adjust model parameters and/or hyperparameters during training. For example, machine learning framework 1335 may be configured to modify model parameters and/or hyperparameters (i.e., hyperparameter tuning) using an optimization technique during training, consistent with disclosed embodiments. Hyperparameters may include training hyperparameters, which may affect how training of a model occurs, or architectural hyperparameters, which may affect the structure of a model. Optimization techniques used may include grid searches, random searches, gaussian processes, Bayesian processes, Covariance Matrix Adaptation Evolution Strategy techniques (CMA-ES), derivative-based searches, stochastic hill-climbing, neighborhood searches, adaptive random searches, or the like.

In some embodiments, machine learning framework 1335 may be configured to generate models based on instructions received from another component of computing system 1300 and/or a computing component outside computing system 1300. For example, machine learning framework 1335 can be configured to receive a visual (e.g., graphical) depiction of a machine learning model and parse that graphical depiction into instructions for creating and training a corresponding neural network. Machine learning framework 1335 can be configured to select model training parameters. This selection can be based on model performance feedback received from another component of machine learning framework 1335. Machine learning framework 1335 can be configured to provide trained models and descriptive information concerning the trained models to model memory 1330.

Any computer program instructions may also be stored in a computer readable medium that can direct one or more hardware processors of a computer, other programmable data processing apparatus, or other devices to function in a particular manner, such that the instructions stored in the computer readable medium form an article of manufacture including instructions that implement the function/act specified in the flowchart or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions that execute on the computer or other programmable apparatus provide processes for implementing the functions/acts specified in the flowchart or block diagram block or blocks.

The embodiments may further be described using the following clauses:
1. A method for configuring a structural-phenotypic model, comprising: obtaining observational data acquired from non-human animals during trials in which the non-human animals were administered at least one of a set of compounds; obtaining structural data for the set of compounds; generating a structural dataset using the structural data by mapping the structural data to a first space; generating a phenotypic dataset using the observational data by mapping the observational data to a second space; and configuring a machine learning model, using the phenotypic dataset and the structural dataset. to map between encoded phenotypic data and encoded structure data.
2. The method of clause 1, wherein the set of compounds comprises one or more of an antidepressant, analgesic, anxiolytic, antipsychotic, cognitive enhancer, hallucinogen, anticonvulsant, mood stabilizer, psychostimulant or ADHD medication, anesthetic, or hypnotic.
3. The method of any one of clauses 1 to 2, wherein the set of compounds comprises one or more reference drugs tested in clinical trials.
4. The method of any one of clauses 1 to 3, wherein the set of compounds comprises one or more preclinical compounds.
5. The method of any one of clauses 1 to 4, wherein mapping the structural data further comprises: determining a pairwise distance between first structure data and second structure data in the structural data, the first structure data corresponding to a first compound in the set of compounds and the second structure data corresponding to a second compound in the set of compounds, and mapping the first compound to a location in the first space using the pairwise distance.
6. The method of clause 5, wherein the pairwise distance comprises a Tanimoto score.
7. The method of any one of clauses 1 to 6, wherein mapping the observational data to the second space further comprises: determining a pairwise distance using first observational data and second observation data in the observational data, the first observation data corresponding to a first compound and the second observational data corresponding to a second compound and associating the first compound with a location in the second space using the pairwise distance.
8. The method of any one of clauses 1 to 7, wherein the pairwise distance comprises at least one of a binary classifier output or a similarity distance.
9. The method of any one of clauses 1 to 8, wherein the observational data is mapped to the second space using a phenotypic machine learning model.
10. The method of clause 9, the method further comprising obtaining, for a subset of the set of compounds, therapeutic class labels, and training the phenotypic machine learning model using the subset and the therapeutic class labels.
11. The method of any one of clauses 1 to 10, further comprising: generating a predicted phenotypic datum using the machine learning model and a structural datum for a trial compound, and providing a predicted therapeutic class based on the predicted phenotypic datum.
12. The method of any one of clauses 1 to 11, wherein generating the phenotypic dataset comprises extracting from the observational data for an animal instant behavioral features describing at least one of size, shape, posture, or movement of the animal.
13. The method of clause 12, wherein generating the phenotypic dataset comprises extracting from the observational data for an animal higher order behavioral features describing at least one of motifs, sequences, or domains of behavioral function of the animal.
14. The method of any one of clauses 1 to 13, wherein generating the phenotypic dataset comprises obtaining a first phenotypic signature for a first compound using first observation data corresponding to the first compound, the first phenotypic signature comprising therapeutic effect probabilities of the compound when administered to a human.
15. The method of clause 14, wherein obtaining the first phenotypic signatures comprises normalizing the first phenotypic signature using a phenotypic signature of a control compound.
16. The method of any one of clauses 1 to 2, wherein generating the phenotypic dataset comprises obtaining phenotypic signatures for a first compound that correspond to minimal and maximal therapeutic doses of the first compound.
17. The method of any one of clauses 1 to 17, wherein generating the phenotypic dataset comprises obtaining phenotypic signatures for a first compound that correspond to minimal and maximal tolerated doses.
18. The method of any one of clauses 1 to 17, wherein the structural data comprises a two-dimensional chemical structure model.
19. The method of any one of clauses 1 to 18, wherein the structural data comprises a three-dimensional chemical structure model.
20. The method of any one of clauses 1 to 19, wherein the structural data comprises a chemical sub-structure model.
21. The method of any one of clauses 1 to 20, wherein the structural data comprises electrostatic data.
22. The method of any one of clauses 1 to 21, wherein the structural data comprises stereochemistry data.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

It is understood that the described embodiments are not mutually exclusive, and elements, components, materials, or steps described in connection with one example embodiment may be combined with, or eliminated from, other embodiments in suitable ways to accomplish desired design objectives.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the invention disclosed herein. It is intended that the specification and examples be considered as exemplary only. It is also intended that the sequence of steps shown in figures are only for illustrative purposes and are not intended to be limited to any particular sequence of steps. As such, those skilled in the art can appreciate that these steps can be performed in a different order while implementing the same method.

What is claimed is:

1. A method for configuring a structural-phenotypic model, comprising:
obtaining observational data acquired from non-human animals during trials in which the non-human animals were administered at least one of a set of compounds;
obtaining structural data for the set of compounds;
generating a structural dataset using the structural data by mapping the structural data to a first space;
generating a phenotypic dataset using the observational data by mapping the observational data to a second space; and
configuring a machine learning model, using the phenotypic dataset and the structural dataset, to map between encoded phenotypic data and encoded structure data.

2. The method of claim 1, wherein the set of compounds comprises one or more of an antidepressant, analgesic, anxiolytic, antipsychotic, cognitive enhancer, hallucinogen, anticonvulsant. mood stabilizer, psychostimulant or ADHD medication, anesthetic, or hypnotic.

3. The method of claim 1, wherein the set of compounds comprises one or more reference drugs tested in clinical trials.

4. The method of claim 1, wherein the set of compounds comprises one or more preclinical compounds.

5. The method of claim 1, wherein mapping the structural data further comprises:
determining a pairwise distance between first structure data and second structure data in the structural data, the first structure data corresponding to a first compound in the set of compounds and the second structure data corresponding to a second compound in the set of compounds, and
mapping the first compound to a location in the first space using the pairwise distance.

6. The method of claim 5, wherein the pairwise distance comprises a Tanimoto score.

7. The method of claim 1, wherein mapping the observational data to the second space further comprises:
determining a pairwise distance using first observational data and second observation data in the observational data, the first observation data corresponding to a first compound and the second observational data corresponding to a second compound and associating the first compound with a location in the second space using the pairwise distance.

8. The method of claim 7, wherein the pairwise distance comprises at least one of a binary classifier output or a similarity distance.

9. The method of claim 1, wherein the observational data is mapped to the second space using a phenotypic machine learning model.

10. The method of claim 9, the method further comprising obtaining, for a subset of the set of compounds, therapeutic class labels, and training the phenotypic machine learning model using the subset and the therapeutic class labels.

11. The method of claim 1, further comprising:
generating a predicted phenotypic datum using the machine learning model and a structural datum for a trial compound, and
providing a predicted therapeutic class based on the predicted phenotypic datum.

12. The method of claim 1, wherein generating the phenotypic dataset comprises extracting from the observational data for an animal instant behavioral features describing at least one of size, shape, posture, or movement of the animal.

13. The method of claim 12, wherein generating the phenotypic dataset comprises extracting from the observational data for an animal higher order behavioral features describing at least one of motifs, sequences, or domains of behavioral function of the animal.

14. The method of claim 1, wherein generating the phenotypic dataset comprises obtaining a first phenotypic signature for a first compound using first observation data corresponding to the first compound, the first phenotypic signature comprising therapeutic effect probabilities of the compound when administered to a human.

15. The method of claim 14, wherein obtaining the first phenotypic signatures comprises normalizing the first phenotypic signature using a phenotypic signature of a control compound.

16. The method of claim 1, wherein generating the phenotypic dataset comprises obtaining phenotypic signatures for a first compound that correspond to minimal and maximal therapeutic doses of the first compound.

17. The method of claim 1, wherein generating the phenotypic dataset comprises obtaining phenotypic signatures for a first compound that correspond to minimal and maximal tolerated doses.

18. The method of claim 1, wherein the structural data comprises a two-dimensional chemical structure model.

19. The method of claim 1, wherein the structural data comprises a three-dimensional chemical structure model.

20. The method of claim 1, wherein the structural data comprises a chemical sub-structure model.

21. The method of claim 1, wherein the structural data comprises electrostatic data.

22. The method of claim 1, wherein the structural data comprises stereochemistry data.
